(19) United States
(12) Patent Application Publication
(10) Pub. No.: US 2020/0022910 A1
(43) Pub. Date: Jan. 23, 2020

(54) PHARMACEUTICAL COMPOSITION OF ORAL SOLUTION OF MUSCARINIC ANTAGONIST

(71) Applicant: FTF Pharma Private Limited, Ahmedabad (IN)

(72) Inventors: Sandip MEHTA, Ahmedabad (IN); Manish UMRETHIA, Ahmedabad (IN); Jayanta Kumar MANDAL, Ahmedabad (IN)

(21) Appl. No.: 16/488,653

(22) PCT Filed: Feb. 23, 2018
(86) PCT No.: PCT/IB2018/051131

§ 371 Date: Aug. 26, 2019

(30) Foreign Application Priority Data
Feb. 24, 2017 (IN) 201721006581

(51) Int. Cl. A61K 9/08; A61K 9/00; A61K 31/4725; A61K 47/14; A61K 47/26; A61K 47/46; A61K 47/12

(52) U.S. Cl. CPC A61K 9/08; A61K 9/0095; A61K 31/4725; A61K 47/12; A61K 47/26; A61K 47/46; A61K 47/14

(57) ABSTRACT

The present invention provides pharmaceutical composition of muscarinic antagonist in oral solution dosage form. The oral solution composition comprises of muscarinic antagonist with other pharmaceutical excipients such as vehicle, preservative, buffering agent, sweetener and flavouring agent. The present invention is an oral solution of muscarinic antagonist that is free from allergic preservatives and having improved stability without risk of over dosage of the drug. The present invention also provides oral solution with flavor that has masked bitter taste of muscarinic antagonist. Further, the present invention also provides a process for preparation thereof.

### FIELD OF INVENTION

The present invention relates to the oral liquid pharmaceutical composition of muscarinic receptor antagonist. More particularly, the present invention relates to oral liquid solution composition comprises of muscarinic receptor antagonist with improved stability and patient compliance. The present invention composition is flavored liquid solution that has masked bitter taste of anti-muscarinic agent.

### BACKGROUND OF THE INVENTION

Anti-muscarinic agent are competitive cholinergic receptor antagonist, selective for the M3 receptor subtype. The binding of acetylcholine to these receptors, particularly M3, plays a critical role in the contraction of smooth muscle, located at neuromuscular junctions in the human bladder detrusor muscle. By preventing the binding of acetylcholine to these receptors, anti-muscarinic agent reduce smooth muscle tone in the bladder, allowing the bladder to retain larger volumes of urine and reducing the number of micturition, urgency and incontinence episodes. The majority of anticholinergic drugs are anti-muscarinic agent.

Important muscarinic antagonist includes but not limited to atropine, hyoscyamine, hyoscine butylbromide, hyoscine hydrobromide, ipratropium, tropicamide, cyclopentolate, pirenzepine, oxybutynin and solifenacin. There are also several subtypes of muscarinic receptors. The human detrusor muscle in bladder contains mainly the M2 and M3 subtypes of muscarinic receptors.

Tolterodine, propiverine, solifenacin, darifenacin, trospium, and fesoterodine are antimuscarinic agent approved for use in overactive bladder (OAB) treatment.

All of the anti-muscarinic drugs used for urinary incontinence are given orally.

EP2902017 discloses pharmaceutical composition of an anti-muscarinic agent with an antioxidant, and a binder in solid dosage form of tablet.

EP2156824 discloses solid formulation in tablet dosage form of crystalline and amorphous solifenacin in mixture with polyethylene glycol (PEG) or polyethylene oxide.

Currently available preparations of anti-muscarinic agent are oral solid preparations (tablets). The oral liquid preparations are more patient compliance as compared to solid dosage form, which is not convenient for all patients to take due to swallowing problem. Hence, it would be preferable to administer active ingredient in liquid solution or suspension dosage form.

EP2572717 discloses a pharmaceutical composition in oral suspension dosage form comprises of an anti-muscarinic agent. But, the suspension dosage sometimes may lead to chances of over or under dosage of the drug.

If product is in a suspension form and patient use it without shaking well, there may be a chance of overdosing or under dosing such patient. Furthermore, overdosing is possible if syringe is filled from the bottom without shaking the bottle. Over-dosage of solifenacin succinate can result in severe anticholinergic effects. In case of severe renal impairment, Cmax, AUC and t_{1/2} are increased to 30%, 100% and 60%, respectively, as compared to normal patients and therefore, should not receive more than 5 mg (5 ml) once daily dose of the drug. In the event of overdose with solifenacin succinate, the patient should be treated with activated charcoal. Gastric lavage is useful if performed within 1 hour, but vomiting should not be induced. Such precautions are necessary to be taken in case of suspension dosage form.

Further, salts of anti-muscarinic s-agent have very high solubility and have strong bitterness and astringency that results a bitter taste and a feeling of numbness in the mouth. Vesicare oral suspension contains methyl parahydroxybenzoate and propyl parahydroxybenzoate. These preservatives may cause allergic reactions.

If product is in a solid dosage form like tablets or capsules, dose adjustment is not possible. Sever renally impaired patient may get exposed to solifenacin due to significantly greater pharmacokinetic parameters e.g. Cmax increased 30%, AUC increased 100%, t½ increased 60%, which may affect safety and efficacy of the product which may require accumulated dose adjustment which is only possible in case of oral liquid solution preparations of the present invention.

Under dosing is also possible if not shaken well. Cmax may fall and compromise efficacy of the product.

To overcome such issues and from the standpoint of the patient safety, development of dosage form of liquid solution has been demanded. Furthermore, in case of solution dosage form, further dose adjustment is very easy with accuracy.

The present invention is directed to liquid solution composition of muscarinic antagonist that has masked bitter taste of muscarinic antagonist. Further, currently inventive formulation is free from allergic preservatives (methyl parahydroxybenzoate, propyl parahydroxybenzoate) and exhibiting improved stability and palatability without risk of over dosage of the drug.

### Object of the Invention

The main object of the present invention is to provide oral pharmaceutical solution of muscarinic antagonist with improved stability and free from allergic preservatives.

Another object of the present invention is to provide oral solution of muscarinic antagonist without risk of over dosage of the drug.

Another object of the present invention is to provide oral pharmaceutical solution of anti-muscarinic agent with flavor that has masked bitter taste of anti-muscarinic agent.

Still another object of the present invention is to provide a process for preparation of oral pharmaceutical solution of muscarinic antagonist.

### SUMMARY OF THE INVENTION

The present invention relates to an oral pharmaceutical solution of muscarinic antagonist with improved stability, free from allergic preservatives and without risk of over dosage of the drug. The present invention also provides oral pharmaceutical solution of anti-muscarinic agent with flavor that has masked bitter taste of anti-muscarinic agent.

Another aspect of the present invention relates to oral solution of muscarinic antagonist that comprises an active ingredient and other pharmaceutically acceptable excipients such as vehicle, preservative, buffering agent, sweetener and flavoring agent. The present invention also provides a process for preparation thereof.

### DETAILED DESCRIPTION OF THE INVENTION

Anti-muscarinic agent block muscarinic receptors and so they are used for treatment of overactive bladder (OAB). They act by reducing the contractile activity of the detrusor muscle in bladder. These drugs act through antagonism at muscarinic M3 receptors located at neuromuscular junctions in the human bladder detrusor muscle.

The present invention relates to an oral pharmaceutical solution of anti-muscarinic antagonist with improved stability free from allergic preservatives and without risk of over dosage of the drug. The present invention also provides oral solution of anti-muscarinic antagonist with flavor that has masked bitter taste of anti-muscarinic agent.

In an embodiment of the present invention, the active pharmaceutical ingredient (API) for oral pharmaceutical solution dosage form is selected from anti-muscarinic agent such as but not limited to atropine, darifenacin, fesoterodine, hyoscyamine, hyoscine butylbromide, hydrobromide, ipratropium, propiverine, solifenacin, tolterodine and trospium. The present invention, more particularly relates to one of the anti-muscarinic agent, solifenacin succinate.

Solifenacinis having an empirical formula of C₂₃H₂₆N₂O₂ and a molecular weight of 480.55, chemically (3R)-1-azabicyclo[2.2.2]octan-3-yl (IS)-1-phenyl-1,2,3,4-tetrahydroisoquinoline-2-carboxylate, and having the chemical structure as follows:

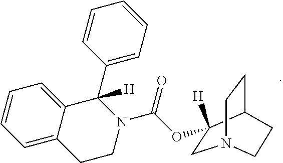 [SMILES: O=C(O[C@H]1CN2CCC1CC2)N1CCc2ccccc2[C@@H]1c1ccccc1 ; "."]

Solifenacin is a medication of an anti-muscarinic class, used for treating contraction of overactive bladder with symptoms of increased urination frequency and urge incontinence. It is a competitive cholinergic receptor antagonist. Solifenacin is M3-receptor selective antagonist which may be more bladder-specific with reduced tendency for anticholinergic side effects. Solifenacin prevents the binding of acetylcholine to this receptor, and reduces smooth muscle tone in the bladder, allowing the bladder to retain larger volumes of urine and reducing the number of micturition, urgency and incontinence episodes. Solifenacin is available in the market under the brand name VESICARE.

The oral solution of the present invention composition comprises solifenacinas an active ingredient, preferably solifenacin succinate and pharmaceutically acceptable excipients. Pharmaceutically acceptable excipients may include vehicle, preservative, buffering agent, sweetener and flavouring agent. The present invention also provides a process for preparation of oral pharmaceutical solution of solifenacin succinate.

Vehicles used in the present pharmaceutical composition are mainly liquid bases which carry active ingredient and other excipients in dissolved. Pharmaceutical vehicles can be classified as aqueous vehicles and oily vehicles. Aqueous vehicles include water, hydro-alcoholic, polyhydric alcohols and buffers. Oily vehicles include vegetable oils, mineral oils, organic oily bases or emulsified bases. Commonly used carrier for oral solution administration can be selected from but not limited to purified water and glycerine.

Preservatives are included in pharmaceutical dosage form and prevent the growth of microorganisms during the product's manufacture and shelf life. Preservatives can be selected from but not limited to propyleneglycol, benzoic acid, potassium sorbate, sodium benzoate, chlorobutanol. In the present invention, preferably sodium benzoate is used as a preservative.

Buffering agent provide stability and pH control to the pharmaceutical formulations. Buffering agent can be selected from but not limited to sodium acetate, sodium citrate, ammonium sulfate, sodium phosphate, disodium hydrogen phosphate, potassium citrate, citric acid monohydrate, trisodium citrate dihydrate.

One or more sweetener (sweetening agent) to be added in liquid formulations that impart sweetness and improve patient compliance through taste masking. The main sweetening agent employed in oral preparations can be selected from but not limited to sucrose, liquid glucose, glycerol, sorbitol, maltitol, saccharin sodium and aspartame. In the present invention, preferably sucralose and liquid maltitol are used as sweetener.

Flavoring agent are added to increase patient acceptance of the drug by masking the specific taste sensations. Flavoring agent can be selected but not limited to essential oils including peppermint oil, orange oil, and lemon oil or can be selected from fruit flavor. In the present invention, preferably peppermint flavor is used.

Below table represents composition of the present invention.

| Sr No | Ingredients | Formula % w/w |
|---|---|---|
| 1 | API | 0.001-10 |
| 2 | Preservative | 0.001-2.0 |
| 3 | Buffering agent | Till suitable pH |
| 4 | Sweetener 1 | 0.025-1.0 |
| 5 | Sweetener 2 | 0.001-70 |
| 6 | Flavouring agent | Q.S. |
| 7 | Vehicle | Q.S. |

The oral pharmaceutical solution of above composition is prepared by following steps but not limited to:
(a) Take required quantity of water
(b) mix one or more excipients till dissolved
(c) adjust desirable pH and
(d) add required quantity of API and mix till it dissolved.

### EXAMPLES

The present invention can be described by way of examples only. They are not to be construed to limit the invention in any manner whatsoever. The following examples are intended to illustrate the various aspects of the invention, though without aiming to limit it.

Below table represents the composition of solifenacin succinate and excipients with its range are shown below:

### Example 1

Solifenacin oral solution 10mg/5ml

| Sr No | Ingredients | Quantity (mg/mL) |
|---|---|---|
| 1 | Solifenacin succinate^{#} | 2.000 |
| 2 | Sodium benzoate^{#} | 0.200 |
| 3 | Sucralose | 1.000 |
| 4 | Liquid maltitol | 400.000 |
| 5 | Peppermint flavor | 0.100 |
| 6 | Citric acid monohydrate^{$} | Q.S. |
| 7 | Purified water | Q.S. to 1 mL |

### Example 2

Solifenacin oral solution 5mg/5ml

| Sr No | Ingredients | Quantity (mg/mL) |
|---|---|---|
| 1 | Solifenacin succinate^{#} | 1.000 |
| 2 | Sodium benzoate^{#} | 0.200 |
| 3 | Sucralose | 1.000 |
| 4 | Liquid maltitol | 400.000 |
| 5 | Peppermint flavor | 0.100 |
| 6 | Citric acid monohydrate^{$} | Q.S. |
| 7 | Purified water | Q.S. to 1 mL |

The oral pharmaceutical solution of above composition is prepared by following steps but not limited to:
(a) Take required quantity of water
(b) mix one or more excipients till dissolved
(c) adjust desirable pH and
(d) add required quantity of API and mix till it dissolved

### Bioequivalence Study

“Bioequivalence study” denotes a scientific basis on which generic and brand name drugs are compared with one another. Drugs are bioequivalent if they enter circulation at the same rate when given in similar doses under similar conditions. Parameters often used in bioequivalence studies are Cₘₐₓ, AUC_{0-infinity}, AUC₀₋ₜ.

The pharmaceutical composition prepared as per the example above was subjected to a bioequivalence study in healthy human subjects. The test was carried out by administering test product with respect to reference product. The test product of the present invention pharmaceutical composition was found bioequivalent to reference product as test product is considered bioequivalent with a reference product, when AUCo₀₋ₜ and Cₘₐₓ is within 80-125% of the reference product, including the 90% confidence interval.

The below table represents the bioequivalence data of the composition of the present invention:

| | Ln-transformed | | | | |
|---|---|---|---|---|---|
| Pharmaco- | Geometric Least Squares Mean | | | 90% Confidence | |
| kinetic Param- | Test | Reference | | Interval | |
| eters (Units) | Product (T) | Product (R) | T/R (%) | Lower | Upper |
| Cₘₐₓ (ng/mL) | 19.0194 | 17.1162 | 111.12 | 105.49 | 117.04 |
| AUC_{0-72 h} | 758.2960 | 697.1407 | 108.77 | 103.98 | 113.79 |
| (ng · hr/mL) | | | | | |

Reference Product (R): VESICARE10 mg film-coated tablet
Test Product (T): Oral solution of Solifenacin succinate (1 mg/ml)

In the present context “Cₘₐₓ” denotes the maximal plasma concentration after administration of the drug; AUC₀₋₇₂ₕ is the area under the plasma concentration versus time curve from time 0 to time 72 hr at steady state conditions.

We claim:

1. A pharmaceutical composition of oral solution of muscarinic antagonist comprises muscarinic antagonist or a pharmaceutically acceptable salt thereof and pharmaceutically acceptable excipients with improved stability.

2. The pharmaceutical composition of oral solution of muscarinic antagonist as claimed in claim 1, wherein the pharmaceutically acceptable excipients are selected from vehicle, preservative, buffering agent, sweetener and flavoring agent.

3. The pharmaceutical composition of oral solution of muscarinic antagonist as claimed in claim 1, wherein muscarinic antagonist comprises atropine, darifenacin, fesoterodine, hyoscyamine, hyoscine butylbromide, hydrobromide, ipratropium, propiverine, solifenacin, tolterodine and trospium.

4. The pharmaceutical composition of oral solution of muscarinic antagonistas claimed in claim 1, wherein the composition is with flavor that has masked bitter taste of muscarinic antagonist.

5. The pharmaceutical composition of oral solution of muscarinic antagonist as claimed in claim 1, wherein the composition is with improved stability and without risk of over dosage of the drug.

6. The pharmaceutical composition of oral solution of muscarinic antagonist as claimed in claim 3, wherein solifenacin salt is solifenacin succinate.

7. A process for preparation of a pharmaceutical composition of oral solution of muscarinic antagonist or a pharmaceutically acceptable salt thereof comprises of following steps:
(a) Take required quantity of water
(b) mix one or more excipients till dissolved
(c) adjust desirable pH and
(d) add required quantity of API and mix till it dissolved
